(19) United States
(12) Patent Application Publication
(10) Pub. No.: US 2026/0091192 A1
(43) Pub. Date: Apr. 2, 2026

(54) METHODS TO INCREASE FUNCTIONAL CAPACITY AND QUALITY OF LIFE IN BRONCHIETASIS PATIENTS WITH NONTUBERCULOUS MYCOBACTERIA

(71) Applicant: Malllinckrodt Pharmaceuticals Ireland Limited, Dublin (IE)

(72) Inventors: James Potenziano, Hazelwood, MO (US); Christopher Miller, Hazelwood, MO (US)

(21) Appl. No.: 19/413,816

(22) Filed: Dec. 9, 2025

Related U.S. Application Data
(63) Continuation of application No. 16/875,000, filed on May 15, 2020.
(60) Provisional application No. 62/849,064, filed on May 16, 2019.

(51) Int. Cl. A61M 16/12; A61M 16/00

(52) U.S. Cl. CPC A61M 16/12; A61M 16/026; A61M 2202/0275

(57) ABSTRACT

Provided herein are method of treating a patient with nontuberculous mycobacteria by administering nitric oxide to the patient for at least 100 days to increase functional capacity and quality of life of the patient.

### FIELD

The present disclosure relates generally to methods to increase functional capacity and quality of life in bronchiectasis patients with nontuberculous mycobacteria.

### BACKGROUND

Non-tuberculous mycobacteria (NTM) are insidious opportunistic organisms that cause lung disease in certain patient populations especially those who have cystic fibrosis (CF) and chronic pulmonary disease. Treatment of NTM, particularly Mycobacterium avium complex (MAC) and Mycobacterium abscessus complex (MABSC), require prolonged multidrug regimens which have a high toxicity and which have suboptimal efficacy because they are inherently antibiotic resistant. Current options for refractory NTM treatment are limited.

As presented herein, the antimicrobial use of inhaled nitric oxide gas (gNO) may be beneficial in this patient population.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Implementations of the present technology will now be described, by way of example only, with reference to the attached figures, wherein:

FIG. 1 is a graph of a six minute walk test which shows significant improvement in exercise over 12 months from non-ambulatory (90 m to 300 m with a SMWT); and

FIG. 2 is a graph of results of a quality of life (QoL) survey showing significant improvement in QoL over 12 months from on-ambulatory (76 to 34 with a validated CRISS Score).

### DETAILED DESCRIPTION

Numerous specific details are set forth in order to provide a thorough understanding of the examples described herein. However, it will be understood by those of ordinary skill in the art that the examples described herein can be practiced without these specific details. In other instances, methods, procedures and components have not been described in detail so as not to obscure the related relevant feature being described. Also, the description is not to be considered as limiting the scope of the embodiments described herein.

Provided herein is a method of treating a patient with nontuberculous mycobacteria. The method may include administering a therapeutic gas that includes nitric oxide to the patient in need thereof for at least 100 days. In an example, the nitric oxide may be administered for at least 200 days. In another example, the nitric oxide may be administered for at least 300 days. In another example, the nitric oxide may be administered for at least one year. In some examples, the patient has cystic fibrosis, bronchiectasis, and/or chronic pulmonary disease.

In yet another embodiment is a method of treating a patient with nontuberculous mycobacteria, wherein the method may include administering a therapeutic gas that includes nitric oxide to the patient in need thereof over a period of at least about 5 days, 7 days, 10 days, 15 days, 30 days, 45 days, 60 days, or 90 days.

The nitric oxide may be administered to a patient in need thereof at a concentration of about 80 ppm, 120 ppm, 160 ppm, 200 ppm, 240 ppm, 280 ppm, 320 ppm, 360 ppm, 400 ppm, 440 ppm, 480 ppm, or 520 ppm. Additionally, the nitric oxide may be administered to a patient in need thereof at a concentration of about 80 to about 500 ppm, about 80 ppm to about 160 ppm, about 160 ppm to 500 ppm, about 80 ppm to about 120 ppm, about 200 ppm to about 320 ppm, about 240 ppm to about 320 ppm, and about 320 ppm to about 500 ppm. The therapeutic gas may be administered at least once daily, twice daily (BID), three times daily (TID), or four times daily. In some examples, each administration of the nitric oxide is for at least 5-7 minutes. In some examples, each administration of the nitric oxide is for at least 10-15 minutes. In some examples, each administration of the nitric oxide is for at least 20 minutes. In some examples, each administration of the nitric oxide is for at least 30 minutes. In some examples, each administration of the nitric oxide is for at least 50 minutes. In other examples, each administration of the nitric oxide is for at least 80 minutes. In some examples, each administration of the nitric oxide is for at least 100 minutes.

In one embodiment, the nitric oxide may be administered to a patient in need thereof at a concentration of about 80 ppm to about 160 ppm. In another embodiment, the nitric oxide may be administered to a patient in need thereof at a concentration of about 160 ppm to about 500 ppm. Optionally, the nitric oxide may be administered to a patient in need thereof at a concentration of about 160 ppm to about 500 ppm, for at least 10-30 minutes for each treatment. Likewise, the nitric oxide may be administered to a patient in need thereof at a concentration of about 80 ppm to about 160 ppm, for at least 50-80 minutes for each treatment. As used herein, the term “about” is understood to mean approximately and may include +/−15% for numerical ranges.

In a further embodiment, the present invention includes a method of treating a patient with nontuberculous mycobacteria, comprising administering a therapeutic gas comprising nitric oxide to the patient at a concentration of at least 150 ppm for an administration of 30-50 minutes, such that the concentration expressed relative per unit time is at least about 3-5 ppm/minute. This concentration expressed relative per unit time is an indication of the surprisingly effective higher dosage or dose load that is permitted according to the present invention. For example, the invention also includes administering a therapeutic gas comprising nitric oxide to the patient at a concentration at least about 300 ppm to about 500 ppm for an administration of about 5 minutes to about 7 minutes, such that the nitric oxide concentration expressed relative per unit time may be at least about 42-60 ppm/minute or at least about 71-100 ppm/minute, respectively. The invention includes but is not limited to administering a therapeutic gas comprising nitric oxide to the patient at a dosage, wherein the therapeutic gas dosage is a concentration expressed relative per unit time, and is at least about 3-5 ppm/minute, 4-7 ppm/minute, 5-10 ppm minute, 10-20 ppm/minute, 20-30 ppm/minute, 30-50 ppm/minute, 40-60 ppm/minute, 50-70 ppm/minute, or 70-100 ppm minute. In another embodiment, the ppm/minute dosage or dose load is selected so as to prevent, minimize, limit, and/or mitigate oxidization of hemoglobin in the patient's bloodstream. Depending on the severity of the nontuberculous mycobacteria infection in the patient's body, a higher or lower ppm/minute of therapeutic gas comprising nitric oxide may be required. Similarly, depending on the risk factors or other comorbidities in the patient, a higher or lower ppm/minute of therapeutic gas comprising nitric oxide may be required. Finally, depending on intelligent dosing factors including blood oxygen content, oxygen dilution, methemoglobin, methemoglobin reductase, CRISS score, or six minute walk performance, a higher or lower ppm/minute of therapeutic gas comprising nitric oxide may be required.

The patient may have increased functional capacity after administration of the therapeutic gas for an extended period of time. For example, the patient may have an improved six minute walk test after administration of the therapeutic gas. In the six minute walk test, the patient walks as far as they are capable of walking in six minutes. In an example, the patient may be able to walk a distance at least twice as far (ex. 100% improvement) in six minutes after administration of the therapeutic gas as compared to prior to administration. In an example, the patient may be able to walk a distance at least three times as far (ex. 200% improvement) in six minutes after administration of the therapeutic gas as compared to prior to administration. In accordance with the present invention, the patient's six minute may be improved by at least about 10%, 15%, 20%, 30%, 40%, 50%, 60%, 70%, 80%, 90%, or 100% after administration of therapeutic gas comprising nitric oxide for an extended period of time comprising at least 10, 30, 60, 90, 100, 200, or 300 days. A “material improvement in quality of life” is herein defined to include an improvement of at least about 10-30% in six minute walk in a patient, including patients having nontuberculous mycobacteria, cystic fibrosis, bronchiectasis, chronic pulmonary disease, or a combination thereof. A “substantially material improvement in quality of life” is herein defined to include an improvement of at least about 30-150% in six minute walk in a patient, including patients having nontuberculous mycobacteria, cystic fibrosis, bronchiectasis, chronic pulmonary disease, or a combination thereof. All instances of the term “patient” includes but is not limited to patients having nontuberculous mycobacteria, cystic fibrosis, bronchiectasis, chronic pulmonary disease, or a combination thereof.

The patient may have improved quality of life after administration of the therapeutic gas for an extended period of time. For example, the patient may have a lower chronic respiratory infection symptom (CRISS) score after administration of the therapeutic gas as compared to prior to administration. The CRISS score is alternatively referred to as the CFRSD-CRISS score by one of skill in the art. A higher CRISS score may be indicative of quality of life symptoms including difficulty breathing, coughing, coughing-up mucus, chest tightness, wheezing, feeling feverish, tiredness, and chills/sweats. A higher CRISS score may be indicative of emotional and activity impacts. For example, emotional impacts may include frustration, sadness/depression, irritability, worry, and difficulty sleeping. Activity impacts include time spent sitting or lying down, reduction of usual activities, and missing school or work. In an example, the patient's CRISS score may be reduced (i.e., improved) by at least about 10%, 15%, 20%, 30%, 40%, 50%, 60%, or 70% after administration of therapeutic gas comprising nitric oxide for an extended period of time comprising at least about 10, 30, 60, 90, 100, 200, or 300 days. A “material improvement in quality of life” is herein defined to include a reduction of at least about 10-30% in CRISS score in a patient, including patients having nontuberculous mycobacteria, cystic fibrosis, bronchiectasis, chronic pulmonary disease, or a combination thereof. A “substantially material improvement in quality of life” is herein defined to include a reduction of at least about 30% in CRISS score in a patient, including patients having nontuberculous mycobacteria, cystic fibrosis, bronchiectasis, chronic pulmonary disease, or a combination thereof. All instances of the term “patient” includes but is not limited to patients having nontuberculous mycobacteria, cystic fibrosis, bronchiectasis, chronic pulmonary disease, or a combination thereof.

The administration of the therapeutic gas reduces the need for co-administration of antibiotics. The discovery of “antibiotic sparing” being possible while treating a patient with nontuberculous mycobacteria by administering a therapeutic gas comprising nitric oxide was highly unexpected. In other words, it has been surprisingly discovered that the therapeutic gas comprising nitric oxide diminishes the amount of infection in the lungs of patient with nontuberculous mycobacteria to a degree that co-administration of antibiotics may be reduced 25-100% for an extended period of time. For clarity, a 100% reduction means a stoppage in antibiotic treatment for the patient. In an example, a patient may not need antibiotics while being administered nitric oxide. In another embodiment, the administration of the therapeutic gas comprising nitric oxide to a patient in need thereof results in a decrease in antibiotics dosing to the patient by 25-100% for at least a period of at least about 7 days, 10 days, 14 days, 20 days, 21 days, 28 days, or 30 days. By reducing the need for co-administration of antibiotics, the administration of the therapeutic gas may also reduce the patient's incidence of antibiotic resistance. Thus, the current invention is also an antibiotic sparing therapeutic strategy.

In another aspect of the present invention, the patient may also have an upregulation of methemoglobin reductase after administration of the therapeutic gas comprising nitric oxide, which in turn reduces the methemoglobin level in the patient over time. Methemoglobin is a type of hemoglobin in the form of metalloprotein, in which the iron in the heme group is in the Fe3+ (ferric) state, not the Fe2+ (ferrous) of normal hemoglobin. Methemoglobin is formed by the reversible oxidation of heme iron to the ferric state. Since ferric iron is unable to bind and transport oxygen, methemoglobin cannot bind oxygen or carry oxygen to organs and tissues in the human body. Methemoglobinemia is a blood disorder in which an abnormal amount of methemoglobin is produced. Methemoglobinemia impairs the human body's ability to deliver oxygen, and may also result in a number of other medical conditions including anemia, congestive heart failure, and chronic obstructive pulmonary disease. The enzyme methemoglobin reductase (alternatively known as NADH-methemoglobin reductase or NADH-cytochrome b5 reductase) is responsible for converting methemoglobin back to hemoglobin and typically limits its accumulation to about 1%-2% or 0-3% of total hemoglobin in the human body of a healthy patient.

In the context of the present invention, the administration of the therapeutic gas comprising nitric oxide to a patient in need thereof may result in an increase in the methemoglobin reductase by at least about 10%, 15%, 20%, 25%, 30%, 40%, 50%, 60%, 70%, 80%, 90%, or 100% relative to the level of methemoglobin reductase in the patient prior to administration of the therapeutic gas. The administration of the therapeutic gas comprising nitric oxide to a patient in need thereof may result in an increase in the methemoglobin reductase by at least a factor of 2 times, 3 times, 4 times, 5 times, 6 times, 7 times, 8 times, 9 times, or 10 times relative to the level of methemoglobin reductase in the patient prior to administration of the therapeutic gas. In another embodiment, the administration of the therapeutic gas comprising nitric oxide to a patient in need thereof results in an increase in the methemoglobin reductase by at least a factor of 3-5 times, 4-6 times, or 5-7 times relative to the level of methemoglobin reductase in the patient prior to administration of the therapeutic gas.

In a further embodiment, administration of the therapeutic gas comprising nitric oxide to a patient in need thereof may result in an increase in the methemoglobin reductase by at least about 10%, 15%, 20%, 25%, 30%, 40%, 50%, 60%, 70%, 80%, 90%, or 100% relative to the level of methemoglobin reductase in the patient at the very beginning of each administration of the therapeutic gas. The administration of the therapeutic gas comprising nitric oxide to a patient in need thereof may result in an increase in the methemoglobin reductase by at least a factor of 2 times, 3 times, 4 times, 5 times, 6 times, 7 times, 8 times, 9 times, or 10 times relative to the level of methemoglobin reductase in the patient at the very beginning of each administration of the therapeutic gas. The increase or improvement in methemoglobin reductase may occur over a span of at least about 10-15 minutes, at least about 20 minutes, at least about 30 minutes, at least about 50 minutes, or at least about 80 minutes during each administration of the therapeutic gas comprising nitric oxide.

In another embodiment, the administration of the therapeutic gas comprising nitric oxide to a patient in need thereof results in an increase in the methemoglobin. Methemoglobin reductase levels may be determined by any validated method or assay known to one of skill in the art. For example, methemoglobin reductase levels may be determined by blood testing by a medical or scientific laboratory, wherein 6.6-13.3 U/g Hb may be indicative of a normal range for an adult patient.

The administration of the therapeutic gas to a patient in need thereof results in an increase in the methemoglobin reductase, and a reduction of total methemoglobin in the patient's blood by at least about 0.25%, 0.50%, 0.75%, 1.00%, 1.25%, 1.50%, 1.75%, 2.00%, 2.25%, 2.50%, 2.75%, 3.00%, 3.25%, 3.50%, 3.75%, 4.00%, 4.25%, 4.50%, 4.75%, 5.00%, 6.25%, 6.50%, 6.75%, 7.00%, 7.25%, 7.50%, 7.75%, 8.00%, 8.25%, 8.50%, 8.75%, 9.00%, 9.25%, 9.50%, 9.75%, or 10.00%. Moreover, the reduction of total methemoglobin in the patient's blood may also occur by at least about 0.25%-2.00%, 2.00%-5.00%, or 5.00%-10.00%. The percentage of methemoglobin is calculated by dividing the concentration of methemoglobin by the concentration of total hemoglobin. Methemoglobin reductase levels may be determined by any validated method or assay known to one of skill in the art, including but not limited to blood testing by a medical or scientific laboratory.

A further aspect of the invention includes intelligent dosing, wherein the patient or medical care provider may safely increase, decrease, or hold constant the dosing of therapeutic gas comprising nitric oxide. To illustrate, the invention includes a method of treating a patient with nontuberculous mycobacteria, comprising: administering a therapeutic gas comprising nitric oxide to the patient for a first period of time of an administration (e.g., at least about 5 minutes, 7 minutes, 10 minutes, 15 minutes, 20 minutes, 30 minutes, 40 minutes, 50 minutes, 60 minutes, 80 minutes, 100 minutes), evaluating one or more intelligent dosing factors including blood oxygen content, oxygen dilution, methemoglobin, methemoglobin reductase, CRISS score, or six minute walk performance; and administering a different amount of nitric oxide gas for a second period of time during the same administration (e.g., at least about 5 minutes, 7 minutes, 10 minutes, 15 minutes, 20 minutes, 30 minutes, 40 minutes, 50 minutes, 60 minutes, 80 minutes, 100 minutes). The intelligent dosing of the treatment of the patient may be occur in two or more administrations over a prolonged period of time including (e.g., at least about 5 days, 7 days, 10 days 15 days, 30 days, 45 days, 60 days, 90 days, 100 days, 200 days, or 300 days). In one embodiment of intelligent dosing, the different amount of nitric oxide gas is confirmed by the user or medical provider. In another embodiment, the invention includes a method of treating a patient with nontuberculous mycobacteria, comprising: administering a therapeutic gas comprising nitric oxide to the patient for a first period of time (e.g., at least about 5 days, 7 days, 10 days, 15 days, 30 days, 45 days, 60 days, 90 days, 100 days, 200 days, or 300 days), evaluating one or more intelligent dosing factors including blood oxygen content, oxygen dilution, methemoglobin, methemoglobin reductase, CRISS score, or six minute walk performance; and administering a different amount of nitric oxide gas for a second period of time (e.g., at least about 5 days, 7 days, 10 days 15 days, 30 days, 45 days, 60 days, 90 days, 100 days, 200 days, or 300 days). Another embodiment of intelligent dosing includes: administering a therapeutic gas comprising nitric oxide to the patient in need thereof at a higher dosage for a shorter period of time; evaluating one or more intelligent dosing factors including blood oxygen content, methemoglobin, methemoglobin reductase, or six minute walk performance; and, ceasing the administration once an intelligent dosing factor threshold is reached. An intelligent dosing factor threshold may include a target blood oxygen content, oxygen dilution, methemoglobin percentage, methemoglobin reductase amount, CRISS score, six minute walk performance, or a combination thereof. An intelligent dosing factor threshold may also include a net improvement in blood oxygen content, oxygen dilution, methemoglobin percentage, methemoglobin reductase amount, CRISS score, six minute walk performance, or a combination thereof. In yet another embodiment. In another embodiment of intelligent dosing, the patient monitoring parameters would change when a target blood oxygen content, oxygen dilution, methemoglobin percentage, methemoglobin reductase amount, CRISS score, six minute walk performance had been reached.

Yet another aspect of the invention includes intelligent dosing, wherein the patient or medical care provider may safely increase, decrease, or hold constant the ppm/minute dosing of the therapeutic gas comprising nitric oxide. To illustrate, the invention includes a method of treating a patient with nontuberculous mycobacteria, comprising: administering a therapeutic gas comprising nitric oxide to the patient for a first ppm/minute for a period of time of an administration (e.g., at of at least about 3-5 ppm/minute, 4-7 ppm/minute, 5-10 ppm minute, 10-20 ppm/minute, 20-30 ppm/minute, 30-50 ppm/minute, 40-60 ppm/minute, 50-70 ppm/minute, or 70-100 ppm minute for a time spanning at least about 5 minutes, 7 minutes, 10 minutes, 15 minutes, 20 minutes, 30 minutes, 40 minutes, 50 minutes, 60 minutes, 80 minutes, 100 minutes), evaluating one or more intelligent dosing factors including blood oxygen content, oxygen dilution, methemoglobin, methemoglobin reductase, CRISS score, or six minute walk performance; and administering a different ppm/minute of therapeutic gas comprising nitric oxide gas for a second period of time during the same administration (e.g., at of at least about 3-5 ppm/minute, 4-7 ppm/minute, 5-10 ppm minute, 10-20 ppm/minute, 20-30 ppm/minute, 30-50 ppm/minute, 40-60 ppm/minute, 50-70 ppm/minute, or 70-100 ppm minute for a time spanning for at least about 5 minutes, 7 minutes, 10 minutes, 15 minutes, 20 minutes, 30 minutes, 40 minutes, 50 minutes, 60 minutes, 80 minutes, 100 minutes).

### Example

A fifty-nine year old female was diagnosed with bronchiectasis in 1988 and Mycobacterium bolletil in 2013. At study onset, CT scan showed multiple bronchiectatic cavities. Medications included IV cefoxitin (6 grams daily), clofazamine, azithromycin, and inhaled Amikacin with significant debilitating side effects.

The subject was non-ambulatory with difficulty speaking, copious secretions, recurrent fevers, and a high subject reported chronic respiratory infection symptom score (CRISS) of 73. She was treated with 160 ppm gNO for 50 minutes, TID for 15 weekdays. On treatment day 15, CRISS score significantly improved by 19 points. The subject was ambulatory, no fevers, with difficulty producing sputum. Culture for M. bolletil decreased from plus 3 to plus 1 over two weeks, which corresponds to a 2-3 log 10 cfu/gm reduction. But at one-week post treatment had increased to plus 2. C-reactive protein decreased from 67 to 24 mg/L. A three-week follow-up found that the patient had a steady improvement in energy level with some return of low-grade fevers, but was otherwise able to speak without shortness of breath and able to ambulate. In subsequent weeks, the patient deteriorated clinically with an increase in NTM bacterial load. At that time a single patient protocol was submitted and approved to move the study to the home and has ensued for over a year now with steady improvement. Subsequently, the subject has received over 900 home-based treatments of gNO without any drug related incidents.

At present, there is a multi-center randomized controlled clinical trial in North America treating CF with gNO (NTC02498535) and two trials to treat patients with NTM pulmonary infections (NTC03331445 and NTC03473314). The patient being investigated in this disclosure is one of the latter studies and is part of an extended and ongoing case evaluation.

Over the last year, an attempt to discontinue antibiotics lasted for 21 days on gNO. Whereas, without gNO the patient lasted 7 days on antibiotics alone.

The subject has received and tolerated BID gNO for 80 minutes for over 200 treatments (Tx).

The subject is the first patient to receive gNO at 160 ppm beyond 100 days (300 Txs). This prolonged investigation may illuminate potential side effects of long-term gNO treatment, as well as identify the physiological impact of gNO on the burden of disease and associated clinical outcomes.

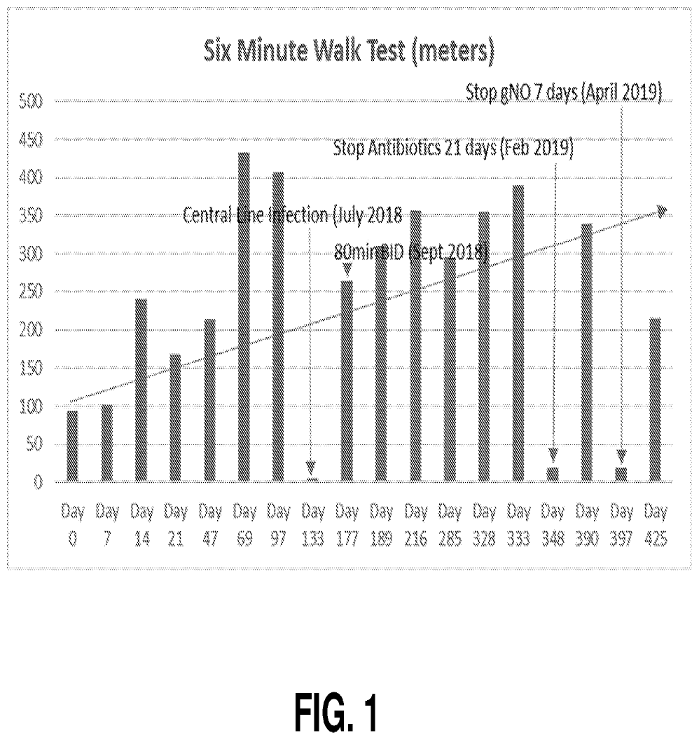

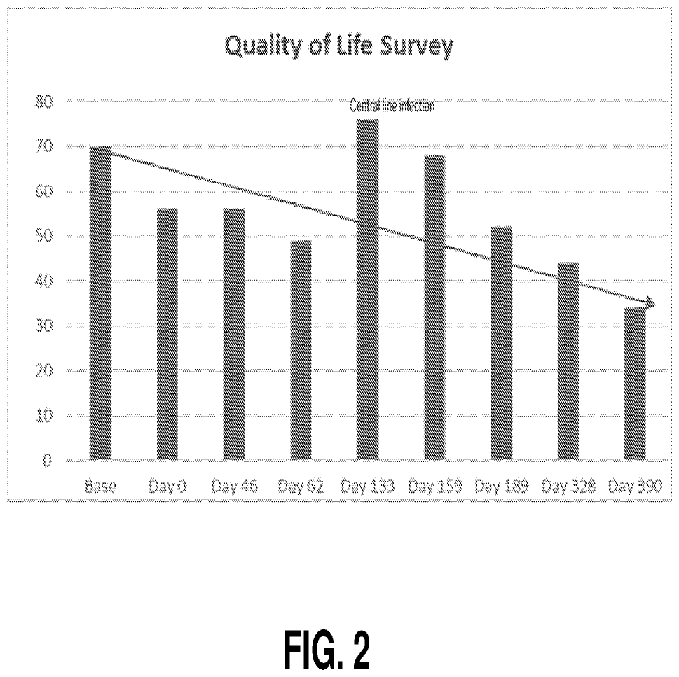

FIG. 1 is a graph of the six minute walk test for the patient, which shows significant improvement in exercise over 12 months from non-ambulatory (90 m to 300 m with a SMWT). FIG. 2 is a graph of results of a quality of life (QoL) survey showing significant improvement in QoL over 12 months from on-ambulatory (76 to 34 with a validated CRISS Score).

The patient showed upregulation of methemoglobin (MetHb) reductase during prolonged exposure to inhaled gNO. Tables 1-12 provide MetHb reductase activity levels at different periods of time and treatment regimens for the subject.

TABLE 1
First week, 30 min; TID for 15 days, Dec. 4, 2017

| Time | | | | | | |
|---|---|---|---|---|---|---|
| (Min) | | | | | Mean | SD |
| 0 | 1 | 0.1 | 0.7 | 0.4 | 0.6 | 0.3 |
| 10 | 2.4 | 2 | 2 | 1.8 | 2.1 | 0.2 |
| 20 | 3.2 | 4.2 | 3.2 | 3 | 3.4 | 0.5 |
| 30 | 3.1 | 5.4 | 4.1 | 3.7 | 4.1 | 0.8 |
| 40 | 4.2 | 7 | 5.6 | 4.9 | 5.4 | 1.0 |
| 50 | 4.6 | | 6.1 | 5.6 | 5.4 | 0.6 |

TABLE 2
Last week, Dec. 22, 2018

| Time | | | | | | |
|---|---|---|---|---|---|---|
| (Min) | | | | | Mean | SD |
| 0 | 0.3 | 0.6 | 0.4 | 0.4 | 0.4 | 0.1 |
| 10 | 1.9 | 1.6 | 1.5 | 1.6 | 1.7 | 0.2 |
| 20 | 2.4 | 2.4 | 2.2 | 2.5 | 2.4 | 0.1 |
| 30 | 3.1 | 3.2 | 2.9 | 3.3 | 3.1 | 0.1 |
| 40 | 3.8 | 4 | 3.7 | 4 | 3.9 | 0.1 |
| 50 | 4.3 | 4.2 | 4.3 | 4.9 | 4.4 | 0.3 |

TABLE 3
First week, 50 min; TID for 90 days, Mar. 9, 2018

| Time | | | | | | |
|---|---|---|---|---|---|---|
| (Min) | | | | | Mean | SD |
| 0 | 0.3 | 0.5 | 0.3 | 0.8 | 0.5 | 0.2 |
| 10 | 1.8 | 1.8 | 2.1 | 2.6 | 2.1 | 0.3 |
| 20 | 2.8 | 2.9 | 2.9 | 3.7 | 3.1 | 0.4 |
| 30 | 3.5 | 4 | 3.6 | 4.6 | 3.9 | 0.4 |
| 40 | 4.5 | 5.1 | 4.5 | 5.4 | 4.9 | 0.4 |
| 50 | 5.2 | 5.2 | 5 | 5.5 | 5.2 | 0.2 |

TABLE 4
Last week, May 28, 2018

| Time | | | | | | |
|---|---|---|---|---|---|---|
| (Min) | | | | | Mean | SD |
| 0 | 0.3 | 0.6 | 0.5 | 0.6 | 0.5 | 0.1 |
| 10 | 1.9 | 1.9 | 1.7 | 1.5 | 1.8 | 0.2 |
| 20 | 2.4 | 2.3 | 2.4 | 2.3 | 2.4 | 0.1 |
| 30 | 3.1 | 3 | 3 | 2.9 | 3.0 | 0.1 |
| 40 | 3.8 | 3.6 | 3.4 | 3.4 | 3.6 | 0.2 |
| 50 | 4.3 | 3.9 | 3.7 | 3.8 | 3.9 | 0.2 |

TABLE 5
Second 90 days, First week, 50 min, TID for 90 days,
Jun. 1, 2018

| Time | | | | | | |
|---|---|---|---|---|---|---|
| (Min) | | | | | Mean | SD |
| 0 | 0.6 | 0.9 | 0.6 | 0.8 | 0.7 | 0.1 |
| 10 | 1.9 | 1.9 | 2.1 | 2 | 2.0 | 0.1 |
| 20 | 2.7 | 2.8 | 3 | 2.6 | 2.8 | 0.1 |
| 30 | 3 | 3.8 | 3.8 | 3.3 | 3.5 | 0.3 |
| 40 | 3.6 | 4.6 | 4.3 | 4.1 | 4.2 | 0.4 |
| 50 | 4.3 | 4.9 | 4.8 | 4.2 | 4.6 | 0.3 |

TABLE 6
Second 90 days, Last week, Aug. 30, 2018

| Time | | | | | | |
|---|---|---|---|---|---|---|
| (Min) | | | | | Mean | SD |
| 0 | 0.5 | 0.6 | 0.7 | 0.6 | 0.6 | 0.1 |
| 10 | 1.9 | 1.4 | 2.2 | 1.5 | 1.8 | 0.3 |
| 20 | 2.8 | 2.1 | 3.1 | 2.3 | 2.6 | 0.4 |
| 30 | 3.1 | 2.9 | 4.1 | 2.9 | 3.3 | 0.5 |
| 40 | 4.9 | 3.7 | 5.4 | 3.4 | 4.4 | 0.8 |
| 50 | 5.5 | 4.3 | 5.8 | 3.8 | 4.9 | 0.8 |

TABLE 7
Third 90 days, First week, 80 min; BID for 90 days,
Sep. 1, 2018

| Time | | | | | | |
|---|---|---|---|---|---|---|
| (Min) | | | | | Mean | SD |
| 0 | 0.7 | 0.4 | 0.4 | 0.7 | 0.6 | 0.2 |
| 10 | 1.7 | 1.7 | 1.6 | 1.9 | 1.7 | 0.1 |
| 20 | 2.6 | 2.2 | 2.8 | 3.4 | 2.8 | 0.4 |
| 30 | 3.2 | 3.3 | 3.5 | 5.3 | 3.8 | 0.9 |
| 40 | 3.6 | 4 | 4.2 | 5.5 | 4.3 | 0.7 |
| 50 | 4.5 | 4.4 | 4.8 | 5.7 | 4.9 | 0.5 |
| 60 | 4.5 | 5 | 6.2 | 7.5 | 5.8 | 1.2 |
| 70 | 5 | 6.3 | 7 | 7.8 | 6.5 | 1.0 |
| 80 | 5.4 | Xxx | 7.4 | Xxx | 6.4 | 1.0 |
| | | (over 8) | | (over 8) | | |

TABLE 8
Third 90 days, Last week, 80 min; BID for 90 days,
Nov. 22, 2018

| Time | | | | | | |
|---|---|---|---|---|---|---|
| (Min) | | | | | Mean | SD |
| 0 | 0 | 0 | 0.1 | 0 | 0.0 | 0.0 |
| 10 | 0.6 | 0.7 | 1.2 | 0.8 | 0.8 | 0.2 |
| 20 | 1.4 | 1.5 | 1.7 | 1.7 | 1.6 | 0.1 |
| 30 | 2.4 | 1.9 | 2.2 | 2.2 | 2.2 | 0.2 |
| 40 | 3 | 2.4 | 2.7 | 2.6 | 2.7 | 0.2 |
| 50 | 3.4 | 3.1 | 3.1 | 3.2 | 3.2 | 0.1 |
| 60 | 3.9 | 3 | 3.5 | 3.7 | 3.5 | 0.3 |
| 70 | 4.3 | 3.2 | 3.9 | 4 | 3.9 | 0.4 |
| 80 | 4.6 | 3.7 | 4.5 | 4.5 | 4.3 | 0.4 |

TABLE 9
Fourth 90 days, First Week, 80 min; BID for 90 days,
Nov. 26, 2018

| Time | | | | | | |
|---|---|---|---|---|---|---|
| (Min) | | | | | Mean | SD |
| 0 | 0 | 0 | 0 | 0.6 | 0.2 | 0.3 |
| 10 | 0.8 | 0.2 | 0.5 | 1.3 | 0.7 | 0.4 |
| 20 | 1.7 | 0.6 | 1.5 | 2.1 | 1.5 | 0.5 |
| 30 | 2.2 | 1.5 | 2.4 | 2.4 | 2.1 | 0.4 |
| 40 | 2.6 | 2.3 | 2.9 | 2.6 | 2.6 | 0.2 |
| 50 | 3.2 | 2.5 | 3.2 | 2.6 | 2.9 | 0.3 |
| 60 | 3.7 | 2.7 | 3.9 | 3.2 | 3.4 | 0.5 |
| 70 | 4 | 3 | 4.5 | 3 | 3.6 | 0.6 |
| 80 | 4.5 | 3.1 | 5.1 | 3.4 | 4.0 | 0.8 |

TABLE 10
Fourth 90 days, Last week, 80 min, BID for 90 days,
Feb. 26, 2019

| Time | | | | | | |
|---|---|---|---|---|---|---|
| (Min) | | | | | Mean | SD |
| 0 | 0 | 0 | 0 | 0 | 0.0 | 0.0 |
| 10 | 0 | 0.8 | 1.2 | 0.7 | 0.7 | 0.4 |
| 20 | 1.6 | 1.4 | 2 | 1.6 | 1.7 | 0.2 |
| 30 | 1.4 | 2.2 | 2.4 | 2.1 | 2.0 | 0.4 |
| 40 | 1.8 | 3.5 | 2.7 | 2.6 | 2.7 | 0.6 |
| 50 | 2.4 | 3.9 | 3.4 | 3.3 | 3.3 | 0.5 |
| 60 | 2.5 | 4.1 | 3.8 | 3.6 | 3.5 | 0.6 |
| 70 | 2.8 | 4.6 | 4.4 | 3.7 | 3.9 | 0.7 |
| 80 | 3.3 | 4.8 | 4.8 | 3.7 | 4.2 | 0.7 |

TABLE 11
Fifth 90 days, First week, 80 min; BID for 90 days,
March 3, 2019

| Time | | | | | | |
|---|---|---|---|---|---|---|
| (Min) | | | | | Mean | SD |
| 0 | 0 | 0 | 0 | 0 | 0.0 | 0.0 |
| 10 | 1.1 | 1 | 1.2 | 0.4 | 0.9 | 0.3 |
| 20 | 1.7 | 2.2 | 2 | 1.4 | 1.8 | 0.3 |
| 30 | 2.5 | 2.9 | 2.8 | 2 | 2.6 | 0.4 |
| 40 | 2.9 | 3.5 | 3.3 | 2.1 | 3.0 | 0.5 |
| 50 | 3.4 | 4.1 | 4.2 | 3.1 | 3.7 | 0.5 |
| 60 | 3.8 | 4.8 | 4.8 | 3.3 | 4.2 | 0.6 |
| 70 | 4.1 | 4.7 | 5.2 | 4.2 | 4.6 | 0.4 |
| 80 | 4.6 | 5.2 | 5.7 | 4.9 | 5.1 | 0.4 |

TABLE 12
Fifth 90 days, Last week, 80 min; BID for 90 days, May 4, 2019

| Time (Min) | | | | Mean | SD |
|---|---|---|---|---|---|
| 0 | 0 | 0 | 0 | 0.0 | 0.0 |
| 10 | 0.1 | 0.9 | 0.5 | 0.5 | 0.3 |
| 20 | 0.8 | 2 | 0.7 | 1.2 | 0.6 |
| 30 | 1.6 | 2.6 | 1.6 | 1.9 | 0.5 |
| 40 | 2.1 | 2.9 | 2.2 | 2.4 | 0.4 |
| 50 | 2.2 | 3.1 | 2.8 | 2.7 | 0.4 |
| 60 | 2.4 | 3.8 | 3.6 | 3.3 | 0.6 |
| 70 | 3 | 3.9 | 3.7 | 3.5 | 0.4 |
| 80 | 3.1 | | 4.2 | 3.7 | 0.5 |

The disclosures shown and described above are only examples. Even though numerous characteristics and advantages of the present technology have been set forth in the foregoing description, together with details of the structure and function of the present disclosure, the disclosure is illustrative only, and changes may be made in the detail, especially in matters of shape, size and arrangement of the parts within the principles of the present disclosure to the full extent indicated by the broad general meaning of the terms used in the attached claims. It will therefore be appreciated that the examples described above may be modified within the scope of the appended claims.

What is claimed is:

1. A method of treating a patient with nontuberculous mycobacteria with intelligent dosing, comprising:
administering a first amount of nitric oxide gas to the patient for a first period of time;
evaluating one or more intelligent dosing factors selected from the group consisting of blood oxygen content, oxygen dilution, methemoglobin, and methemoglobin reductase; and
administering a second amount of nitric oxide gas for a second period of time based on the one or more intelligent dosing factors.

2. The method of claim 1, wherein the first amount of nitric oxide gas is greater than the second amount of nitric oxide gas.

3. The method of claim 1, wherein the first amount of nitric oxide gas is less than the second amount of nitric oxide gas.

4. The method of claim 1, wherein the one or more intelligent dosing factors includes methemoglobin.

5. The method of claim 1, wherein the first period of time is about 5 minutes to about 100 minutes.

6. The method of claim 1, wherein the second period of time is about 5 minutes to about 100 minutes.

7. The method of claim 1, further comprising administering a dose of an antibiotic treatment while administering the first amount of nitric oxide gas and/or the second amount of nitric oxide gas.

8. The method of claim 7, wherein the dose of the antibiotic treatment is reduced from what would be administered without administration of nitric oxide gas.

9. The method of claim 1, wherein the first amount of nitric oxide gas is about 80 ppm to about 500 ppm.

10. The method of claim 9, wherein the first amount of nitric oxide gas is about 80 ppm to about 160 ppm.

11. The method of claim 1, wherein the second amount of nitric oxide gas is about 80 ppm to about 500 ppm.

12. The method of claim 11, wherein the second amount of nitric oxide gas is about 200 ppm to about 500 ppm.

13. The method of claim 1, wherein the two administering steps each occur more than once over a prolonged period of time of at least 5 days.

14. The method of claim 13, wherein the prolonged period of time is at least 100 days.

15. The method of claim 13, wherein the prolonged period of time is at least 200 days.

16. The method of claim 13, wherein the prolonged period of time is at least 300 days.

17. The method of claim 1, further comprising ceasing administration of nitric oxide gas when an intelligent dosing factor threshold is reached.

18. The method of claim 1, further comprising evaluating a CRISS score and/or six minute walk performance of the patient.

19. The method of claim 18, wherein the patient has a reduced CRISS score by 50% after administration of the nitric oxide gas compared to a baseline.

20. The method of claim 18, wherein the patient has an improved six minute walk performance by at least 10% after administration of the nitric oxide gas compared to a baseline.
